(19) United States
(12) Patent Application Publication
(10) Pub. No.: US 2024/0277902 A1
(43) Pub. Date: Aug. 22, 2024

(54) METHOD FOR MANUFACTURING PERIPHERAL NERVE-MIMICKING MICROTISSUE AND USES THEREOF

(71) Applicant: INNOSTEM BIO, Busan (KR)

(72) Inventors: Young-Il YANG, Busan (KR); Won Jin LEE, Busan (KR); Chung Eun YEUM, Busan (KR)

(73) Assignee: INNOSTEM BIO, Busan (KR)

(21) Appl. No.: 18/568,851

(22) PCT Filed: Jun. 7, 2022
(86) PCT No.: PCT/KR2022/007991

§ 371 Date: Dec. 11, 2023

(30) Foreign Application Priority Data
Jun. 11, 2021 (KR) 10-2021-0075824

(51) Int. Cl. A61L 27/38; A61L 27/54; C12N 5/079

(52) U.S. Cl. CPC A61L 27/3878; A61L 27/3834; A61L 27/3895; A61L 27/54; C12N 5/0618; A61L 2300/412; A61L 2430/32; C12N 2513/00; C12N 2533/52; C12N 2533/54

(57) ABSTRACT

The present invention relates to a method for manufacturing a peripheral nerve-mimicking microtissue and to uses thereof, and relates to a method for manufacturing a peripheral nerve-mimicking microtissue having a diameter of 100±20 μm composed of about 100 to 500 cells, comprising isolating an culturing peripheral nerve-derived stem cells (PNSCs), and forming a cell-to-cell and cell-to-extracellular matrix binding through suspension culture of the isolated and cultured PNSCs, wherein the microtissue produced by culturing in a suspended culture environment has structural properties in which about 100 to 500 cells are assembled through cell-to-cell binding by β-catenin, the extracellular matrix (ECM) produced and secreted by the PNSCs between cells accumulates, and binding is performed by β1-integrin between the accumulated ECM and cells, and this is similar to the peripheral nerve composition and constituent cells that are regenerated after injury. Functionally, the present invention can induce nerve tissue regeneration by secreting neurotrophic agents that act centrally on nerve regeneration in the peripheral nerve-mimicking microtissue.

### TECHNICAL FIELD

The present disclosure relates to a method of manufacturing a peripheral nerve-mimicking microtissue and a use thereof.

### BACKGROUND ART

After being damaged, neural tissue causes complications such as an irreversible, permanent loss of motor, sensory, and autonomic functions. As a result, nerve damage results in serious social, economic, and medical issues. In the case of spinal cord injury which is one of the most common nerve injuries, there are about 200,000 patients in the United States, estimated that around 10,000 new cases occur every year. Depending on the severity, treatment costs billions of won per patient and about 200 million won per year, making it a representative incurable disease with high unmet economic and social needs. In Korea, investigation revealed that there are about 70,000 patients with permanent spinal cord injury.

Nerve damage is categorized into primary and secondary damage, with primary damage caused by damage in nerve tissues and blood vessels due to physical compression by external injury. Secondary damage occurs due to inflammatory reactions, free oxygen, free radicals, ischemia and hypoxia, edema, and cell death following the primary damage. The only treatment includes high dose administration of corticosteroid within 1 day after injury, surgical removal of damaged tissues, hematoma, and bones that compress the nerve tissue, and spinal fixation. Currently, there are no therapeutic agents that may prevent and alleviate secondary damage following the primary damage and regenerate damaged nerves.

A stem cell therapeutic agent is a biopharmaceutical applicable to intractable diseases that cannot be treated with conventional therapeutic agents, such as nerve damage. Stem cell therapeutic agents have shown mechanisms and effectiveness in alleviating and suppressing secondary damage that occurs after nerve damage, and have been found to mediate the regenerative effect after nerve damage through an indirect mechanism through anti-inflammation, immunoregulation, anti-radical, cytoprotection, angiogenesis, and secretion of neurotrophic factors, and a direct mechanism that promotes the growth and production of axons and myelin by differentiating directly into neuron cells or oligodendrocytes.

A mechanism has been proposed that stem cell therapeutic agents for neuroregeneration enhances regeneration by secreting neurotrophic factors from transplanted cells. Major neurotrophic factors (NFs) that play a major role in neuroregeneration include brain-derived neurotrophic factor (BDNF), ciliary neurotrophic factor (CNTF), glial-derived neurotrophic factor (GDNF), nerve growth factor (NGF), and neurotrophin-3 (NT-3). Neurotrophic factors activate the intrinsic tissue regeneration mechanism of surrounding cells after nerve damage. A mechanism has been proposed that the neurotrophic factor secreted from the engrafted cell therapeutic agents after transplantation binds to Trk receptors or p75^{NTR} receptor-positive cells around the affected area to produce neuronal survival and axons and regenerate by activating the neuronal intrinsic tissue homeostatic mechanism. It has been reported that BDNF and NGF play a major role in the cytoprotection and axon-generation of rubrospinal and adrenergic/sensory neurons.

Stem cells that are dominantly used as therapeutic agents for nerve damage are bone marrow (BM), cord blood (CB) and umbilical cord (UC), and adipose tissue (AT)-derived mesenchymal stem cells (MSCs), with ongoing preclinical or clinical research conducted by applying central nervous system-derived neural stem cells (NSCs), embryonic stem cell or induced pluripotent stem cell-derived NSCs, or oligodendrocyte progenitor cells (OPCs) as stem cell therapeutic agents. Skin or hair follicle-derived progenitor cells are stem cells present in the tissues of the peripheral nervous system with an ability to differentiate into Schwann cells, neuron cells, and mesenchymal cells, have characteristics similar to neural crest-derived cells that exist temporarily during the development, and are applied as a cell source for a regenerative therapy after nerve damage. However, in order to maximize neuroregenerative functions of stem cell therapeutic agents, technologies capable of increasing gene expression of neurotrophic factors and protein secretion are required.

Artificial gene introduction may increase expression of neurotrophic genes and protein secretion of stem cells, but safety issues due to artificial manipulation may be an obstacle to clinical application. However, by incorporating 3D culture technology, it is possible to increase the cell intrinsic genetic function by providing a 3D environment to cells. By providing a three-dimensional (3D) environment similar to in vivo through 3D culture, cell-to-cell and cell-to-extracellular matrix (ECM) interactions are enabled, and as a result, mechanisms for expression and production of genes and proteins in the cell may be increased. Porous supports or hydrogels are widely used to provide the 3D environment. In the case of constructing a 3D structure by seeding cells in such a biomaterial, licensing issues may arise when such the structure and cells are co-transplanted, such that a technology that allows cells to form a 3D structure without assistance by a support or hydrogel is required.

### DISCLOSURE OF THE INVENTION

### Technical Goals

An object of the present disclosure is to provide a method of manufacturing a peripheral nerve-mimicking microtissue which has cell-to-cell and cell-to-ECM bindings of 100-500 cells formed by β-catenin and integrin-β1 by culturing adult peripheral nerve-derived stem cells (PNSCs) in a suspension culture environment while peripheral nerve-specific ECM produced and secreted by PNSCs accumulates in cell matrix, and is capable of inducing neuroregeneration by secreting any one or more neurotrophic family factors selected from the group consisting of BDNF, NGF, neutrophin-3, and neurotrophin-4, which are neurotrophic factors produced and secreted by PNSC: ephrin family factors: any one or more GDNF family factors selected from the group consisting of GDNF and artemin: or any one or more CNTF family factors selected from the group consisting of IL-6, CNTF, and LIF.

In addition, another object of the present disclosure is to provide a peripheral nerve-mimicking microtissue which is a spheroid cell structure bound with 100 to 500 PNSCs that are subjected to suspension culture in a culture medium in which human serum albumin (HAS), dexamethasone (DEX), and N-acetylcysteine (NAC) are included, has a diameter of 100±20 μm, and is formed by cell-to-cell bindings of PNSCs and PNSC-to-extracellular matrix (ECM) bindings.

In addition, another object of the present disclosure is to provide a pharmaceutical composition for treating nerve damage diseases, including the peripheral nerve-mimicking microtissue as an active ingredient.

In addition, another object of the present disclosure is to provide a pharmaceutical composition for treating neuroinflammatory diseases, including the peripheral nerve-mimicking microtissue as an active ingredient.

### Technical Solutions

In order to achieve the above objects, the present disclosure provides a method of manufacturing a peripheral nerve-mimicking microtissue, including: 1) monolayer-culturing peripheral nerve-derived stem cells (PNSCs); and 2) collecting the monolayer-cultured PNSCs to be subjected to suspension culture in a culture medium in which human serum albumin (HSA), dexamethasone (DEX), and N-acetylcysteine (NAC) are included.

In addition, the present disclosure provides a peripheral nerve-mimicking microtissue which is a spheroid cell structure bound with 100 to 500 PNSCs that are subjected to suspension culture in a culture medium in which HSA, DEX, and NAC are included, has a diameter of 100±20 μm, and is formed by cell-to-cell bindings of PNSCs and PNSC-to-extracellular matrix (ECM) bindings.

In addition, the present disclosure provides a pharmaceutical composition for treating nerve damage diseases, including the peripheral nerve-mimicking microtissue as an active ingredient.

In addition, the present disclosure provides a pharmaceutical composition for treating neuroinflammatory diseases, including the peripheral nerve-mimicking microtissue as an active ingredient.

### Advantageous Effects

The present disclosure relates to a method of manufacturing a peripheral nerve-mimicking microtissue and a use thereof, and particularly, to a method of manufacturing a peripheral nerve-mimicking microtissue which is made up of 100 to 500 cells in a diameter of 100±20 μm, obtained by isolating and culturing peripheral nerve-derived stem cells (PNSCs) and then forming cell-to-cell and cell-to-extracellular matrix bindings using the isolated and cultured PNSCs via suspension culture. The microtissue manufactured by culture in a suspension culture environment has structural characteristics in which 100 to 500 cells are self-assembled through cell-to-cell bindings by β-catenin, extracellular matrix (ECM) produced and secreted by PNSC between cells is accumulated, and the accumulated ECM and cells are bound by β1-integrin. The constituent cells include an immature peripheral nerve-derived stem cell, as well as a Schwann progenitor cell, a repair Schwann cell, a myelinating Schwann cell, and an interstitial stromal cell. This is similar to the peripheral nerve composition and constituent cells that regenerate after damage. Functionally, it is possible to induce neuron tissue regeneration by secreting neurotrophic factors that functionally play a pivotal role in neuroregeneration in peripheral nerve-mimicking microtissues.

### BRIEF DESCRIPTION OF DRAWINGS

FIG. 1 shows a method of manufacturing a peripheral nerve-mimicking microtissue for neuroregeneration from peripheral nerve-derived stem cells (PNSCs).

FIG. 2 shows a correlation of size according to the number of PNSCs that make up peripheral nerve-mimicking microtissues.

FIG. 3 shows methods of controlling a production frequency and size of a microtissue according to a concentration of human serum albumin (HSA) added in a suspension culture medium.

FIG. 4 shows methods of controlling a size of a peripheral nerve-mimicking microtissue through adjustment of a seeding density of PNSCs.

FIG. 5 shows results that accumulation of radical oxygen species (ROS, free radicals, active oxygen) in microtissues increases relative to a size.

FIG. 6 shows a cell survival rate according to a size of a microtissue.

FIG. 7 shows cytoprotective effects of N-acetyl cysteine (NAC) and dexamethasone (DEX) on constituent cells in a microtissue.

FIG. 8 shows cytoprotective mechanisms of constituent cells in a microtissue by NAC and DEX.

FIG. 9 shows structural characteristics of a peripheral nerve-mimicking microtissue manufactured in a culture environment for industrial-level production.

FIG. 10 shows effects in which a Wnt signaling pathway in a peripheral nerve-mimicking microtissue is enhanced.

FIG. 11 shows structural characteristics of cells making up a peripheral nerve-mimicking microtissue manufactured in a culture environment for industrial-level production.

FIG. 12 shows characteristics of neurotrophic gene expression of a peripheral nerve-mimicking microtissue manufactured in a culture environment for industrial-level production.

FIG. 13 shows a result of evaluating cell damage by ROS in a peripheral nerve-mimicking microtissue.

FIG. 14 shows results of comparing an expression rate of cell death regulating factors in a peripheral nerve-mimicking microtissue.

FIG. 15 shows results of identifying significantly high cell survival rate and low annexin V expression in a microtissue compared to PNSCs.

FIG. 16 shows a result of identifying that an ability of PNSCs to secrete neurotrophic proteins is enhanced through microtissue formation.

FIGS. 17 to 20 show results of comparative evaluation of neuroactivity, anti-inflammatory, and neovascularization inducing titers by cell-based analysis using conditioned media obtained from microtissues and PNSCs.

FIG. 21 shows a result of evaluation on a survival rate and efficacy after transplantation of peripheral nerve-mimicking microtissues in vivo.

FIG. 22 shows a result of evaluation on a content of neurotrophic factors in the spinal cord a week after administration of PNSCs or microtissues.

FIG. 23 shows results of identifying myelination and axon growth through administration of PNSCs or microtissues, compared to animals without administration.

FIG. 24 shows results in that myelination and axon growth are significantly high when microtissues are administered compared to PNSCs.

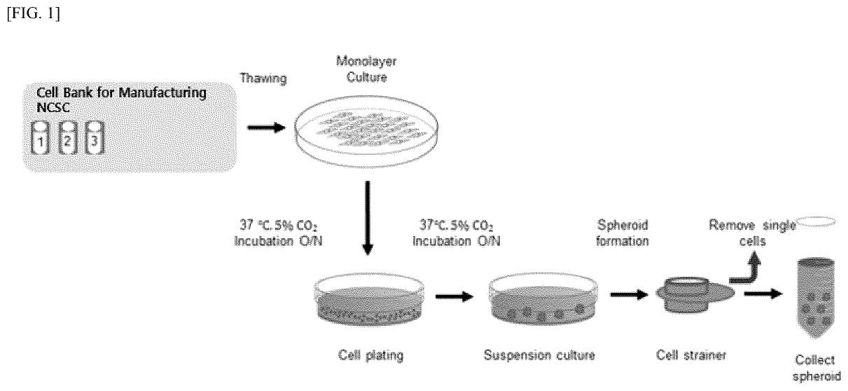

### BEST MODE FOR CARRYING OUT THE INVENTION

The present disclosure provides a method of manufacturing a peripheral nerve-mimicking microtissue including: 1) monolayer-culturing peripheral nerve-derived stem cells (PNSCs); and 2) collecting the monolayer-cultured PNSCs to be subjected to suspension culture in a culture medium in which human serum albumin (HSA), dexamethasone (DEX), and N-acetylcysteine (NAC) are included.

Preferably, in the suspension culturing, 0.25 to 2.5×10⁵ PNSCs per cm² area of a culture vessel may be seeded, but it is not limited thereto.

Preferably, the culture medium may include, but is not limited to, 0.01 to 1% HSA, 0.1 to 5 μM DEX, and 0.1 to 10 mM NAC.

Preferably, the suspension culture may induce cell-to-cell bindings of the PNSCs.

Preferably, the peripheral nerve-mimicking microtissue is a spheroid cell structure with 100 to 500 PNSCs bound together, and may have a diameter of 100±20 μm, but is not limited thereto.

The present disclosure provides a peripheral nerve-mimicking microtissue which is a spheroid cell structure bound with 100 to 500 PNSCs that are subjected to suspension culture in a culture medium including HSA, DEX, and NAC, has a diameter of 100±20 μm, and is formed via cell-to-cell bindings of PNSCs and PNSC-to-extracellular matrix (ECM) bindings.

Preferably, the peripheral nerve-mimicking microtissue has a structure in which collagen type-VI and laminin produced and secreted from PNSCs are accumulated in intercellular space and cells are bound to extracellular matrix by CD29 and cells are bound to cells by β-catenin.

Preferably, the peripheral nerve-mimicking microtissue may include a peripheral nerve-derived adult stem cell, a Schwann progenitor cell, a repair Schwann cell, a myelinating Schwann cell, and a mesenchymal interstitial stromal cell, and more preferably, a GFAP−/S100β−/Sox10+ undifferentiated neural crest cell, a GFAP+/S100β+/myelin+ myelinated Schwann cell, a GFAP+/GAP43+/myelin-repair Schwann cell, and a GFAP−/CD140a+ interstitial stromal cell, but is not limited thereto.

Preferably, the peripheral nerve-mimicking microtissue may have a Wnt/β-catenin signaling pathway activated.

Preferably, the peripheral nerve-mimicking microtissue may have increased expression of, but is not limited to, any one or more neurotrophic factors selected from the group consisting of BDNF, EFNA1, EFNA2, EFNA3, EFNA4, EFNA5, EFNB1, EFNB2, EFNB3, CTNF, GDNF, LIF, NGFB, NTF3, NTF5, NRG1, NRG2, NRG3, NRG4, and ZFP91; any one or more growth factors selected from the group consisting of EGF, FGF1, FGF2, FGF4, FGF5, FGF6, FGF7, FGF8, FGF9, FGF10, FGF11, FGF12, FGF13, FGF14, FGF16, FGF18, FGF19, FGF20, FGF23, IGF1, and GAS6; any one or more immune response regulating factors selected from the group consisting of CLC, CTF1, CSF1, CSF2, CSF3, GH1, GH2, FLT3LG, IDO1, IL2, IL3, IL5, IL7, IL9, IL10, IL11, IL12A, IL12B, IL15, IL20, IL21, IL22, IL23A, IL24, IL26, IL28A, IL29, IFNA1, IFNB1, IFNW1, IFNK, IFNE1, IFNG, KITLG, LEP, PRL, TGFB, TPO, and TSLP: or any one or more angiogenesis inducing factors selected from the group consisting of ANGPT1, ANGPT2, ANGPT4, EFNA1, EFNA2, EFNA3, EFNA4, EFNA5, EFNB3, EPO, PDGFC, PDGFD, VEGFA, VEGFB, and VEGFC.

In addition, the present disclosure provides a pharmaceutical composition for treating nerve damage diseases, including the peripheral nerve-mimicking microtissue as an active ingredient.

Preferably, the pharmaceutical composition may promote regeneration of nerve tissues, but is not limited thereto.

In addition, the present disclosure provides a pharmaceutical composition for treating neuroinflammatory diseases, including the peripheral nerve-mimicking microtissue as an active ingredient.

More specifically, the peripheral nerve-mimicking 3D microtissue according to the present disclosure is characterized by the composition, structure, and biological components as described below, and provides a basis for application as a neuroregenerative therapeutic agent.

(1) The peripheral nerve-mimicking microtissue has a spheroid cell structure with 100 to 500 PNSCs cells bound together in a diameter of 100±20 μm.

(2) The peripheral nerve-mimicking microtissue includes a peripheral nerve-derived adult stem cell, a Schwann progenitor cell, a repair Schwann cell, a myelinating Schwann cell, and a mesenchymal interstitial stromal cell.

(3) The peripheral nerve-mimicking microtissue has laminin and collagen type-VI accumulated, which are peripheral specific extracellular matrices produced and secreted from PNSCs in the cell matrix, and shows structural characteristics with enhanced structural stability through cell-to-cell and cell-to-ECM bindings by β-catenin and integrin-β1.

(4) The peripheral nerve-mimicking microtissue has biological properties in that secretion of neurotrophic factors in constituent cells increases due to enhanced structural stability. The peripheral nerve-mimicking microtissue has a microenvironment that allows cell-to-cell and cell-to-extracellular matrix interactions, enabling activation of Wnt/β-catenin and integrin-β1/FAK signaling pathways to induce an increase in expression of downstream target genes. As a result, compared to PNSCs, peripheral nerve-mimicking microtissues is a microtissue in which peripheral nerve-specific ECMs accumulate, expression of neurotrophic factors such as artemin, BDNF, CNTF, GDNF, IGF, IL-6, NGF, and NT-3 mRNA that are produced and secreted by PNSCs increases, and protein secretion may increase.

(5) The peripheral nerve-mimicking microtissue may activate the neuroregenerative mechanism through secretion of neurotrophic factors including active ingredients such as artemin, BDNF, CNTF, GDNF, IGF, NGF, and NT-3 to induce a mechanism to promote neurons, axon regeneration, and myelination in the damaged area.

(6) Finally, compared to the PNSCs, the peripheral nerve-mimicking microtissue may enhance neurotrophic gene expression and protein secretion, enhance a neuroregenerative biological mechanism of PNSCs, and increase the neuroregenerative effect compared to PNSCs.

### Modes for Carrying Out the Invention

Hereinafter, the present disclosure will be described in detail through example embodiments. These example embodiments are merely for the purpose of describing the present disclosure in more detail, and it will be apparent to a person skilled in the art that the scope of the present disclosure is not limited by these example embodiments, according to the gist of the present disclosure.

### Example 1

The present Example provides a method of controlling a size of a microtissue according to the number of PNSCs that make up the peripheral nerve-mimicking microtissue. PNSCs collected from a monolayer culture environment are suspended in a suspension culture medium at a density of 1.0E+06/mL. The suspension culture medium is prepared by adding 1% human serum albumin (HSA, Green Cross Corporation), 1 μM dexamethasone, and 1 mM N-acetylcysteine in DMEM/F12 culture media. PNSCs seeded using ultra low attachment (ULA) culture vessel induce cell-to-cell bindings by preventing adhesion to the culture vessel. For PNSCs suspended in suspension culture medium, 100, 200, 500, 750, 1000, 2500, and 5000 cells are seeded in each well in a 96-well ULA culture vessel (SPL Life Sciences, Seoul, Korea) and then centrifuged at 500× for 10 minutes to collect cells in the center of the culture vessel. Afterwards, photographs were taken on the 1^{st}, 2^{nd}, and 3^{rd} day of culture, and the diameter of the microtissue was measured by a photoplanimetry method using ImageJ (NIH, Bethesda, MD).

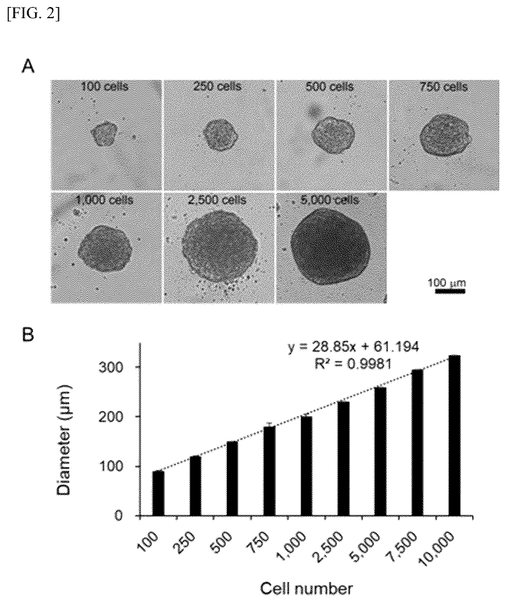

In FIG. 2A, PNSCs seeded in ULA 96-well culture vessels were not adhered to the surface of the culture vessel, and seeded cell-to-cell aggregation and assembly were formed within 2 hours of culture, after which the microtissue morphology formed during the entire culture period was maintained. It may be seen that a size increases proportional to the number of cells that make up the peripheral nerve-mimicking microtissue. As presented in FIG. 2B, a coefficient of correlation (R) value for a size of the microtissue and the number of consisting PNSCs is 0.99, suggesting a method of controlling the size of peripheral nerve-mimicking microtissue by controlling the number of cells seeded. However, the size of the microtissues formed did not change over the culture period.

### Example 2

The Example provides a method of controlling the formation, number, and size of the peripheral nerve-mimicking microtissue by controlling a concentration of human serum albumin (HSA) added in the suspension culture medium. PNSCs collected from the monolayer culture environment are suspended in a suspension culture medium at a density of 1.0E+06/mL. The suspension culture medium was prepared by adding 1 μM dexamethasone and 1 mM N-acetylcysteine in a DMEM/F12 culture medium. ULA 6-well culture vessel (SPL Life Sciences) was used for preparation of peripheral nerve-mimicking microtissues. 1.0×10⁵ PNSCs per cm² area were seeded in ULA 6-well culture vessels. 3 mL of suspension culture medium including 0, 0.01, 0.1, and 1% HSA was added to the culture vessel and subjected to suspension culture for 24 hours. The number and size of the formed peripheral nerve-mimicking microtissue were measured by a photoplanimetry method using ImageJ.

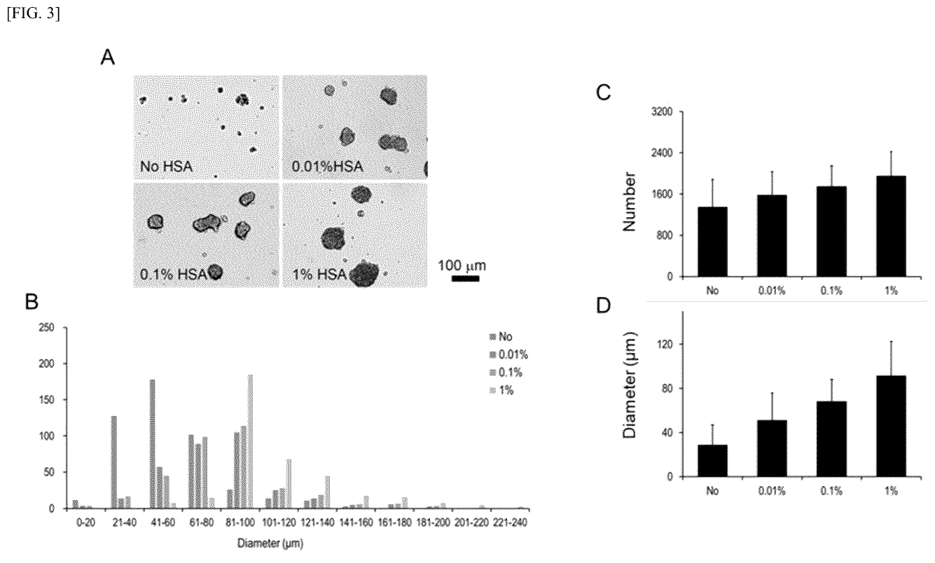

Shown in FIG. 3A is the difference in the formation frequency and size distribution of microtissues according to the concentration of HSA in the suspension culture medium. The number and size of microtissues formed under the conditions cultured using culture medium without HSA and culture medium with 0.01, 0.1, and 1% HSA added were measured using ImageJ. As shown in FIGS. 3B-3D, the formation frequency of microtissue formation increased significantly with the addition of HSA compared to a group without HSA. In the HSA-added group, no significant difference was found in the frequency of microtissues formed according to HSA concentration. However, it was found that the size of peripheral nerve-mimicking microtissue increased significantly in proportion to the concentration of HSA added, suggesting that formation and size of the microtissue may be controlled through HSA addition and the control of concentration added in a suspension culture environment.

### Example 3

The Example provides a method of controlling the number and size of peripheral nerve-mimicking microtissues by adjusting a density of PNSC seeded per culture vessel area for the industrial mass production of the peripheral nerve-mimicking microtissue. PNSCs collected from the monolayer culture environment are suspended in a suspension culture medium at a density of 1.0E+06/mL. The suspension culture medium is prepared by adding 1% human serum albumin (HSA, Green Cross Corporation), 1 μM dexamethasone, and 1 mM N-acetylcysteine in DMEM/F12 culture media. ULA 6-well culture vessels (SPL Life Sciences) were used for preparation of peripheral nerve-mimicking microtissues. 2.5, 5.0, 7.5, 10.0, 15.0×10⁴ PNSCs per cm² area were seeded in the culture vessel, and 3 mL of a suspension culture medium was added, followed by culture for 24 hours. The number and size of microtissues formed were measured using ImageJ.

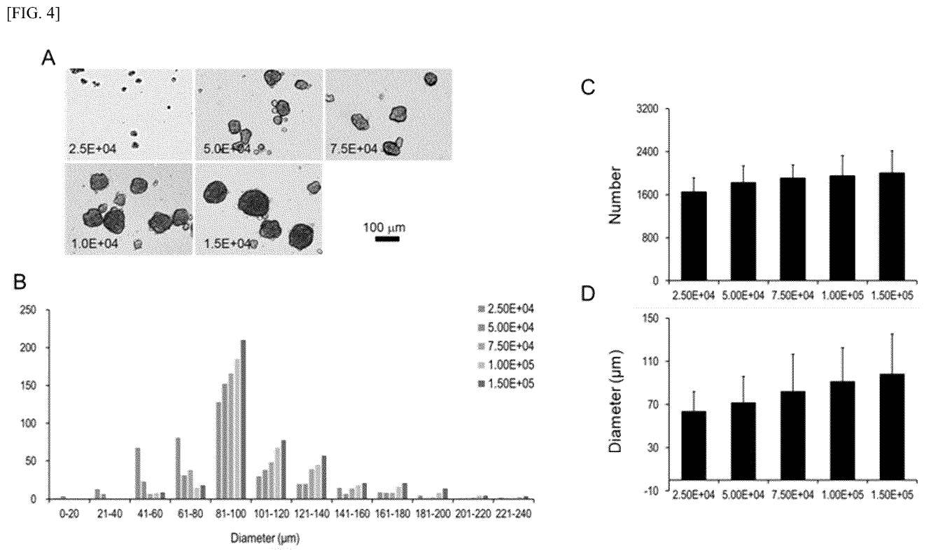

FIG. 4 presents results of controlling a size of peripheral nerve-mimicking microtissues by adjusting PNSC seeding density. Although there was no significant difference in the density of PNSC seeded per unit area, that is, it was possible to determine the correlation, in terms of the number of microtissues formed according to the number of cells that the size of microtissues increases with the density of cell seeded. In order to prepare peripheral nerve-mimicking microtissues in a diameter of 100±20 μm, it is possible to determine the conditions for suspension culture by seeding cells at a density of 1.0˜1.5×10⁵ per cm².

### Example 4

As the size of microtissues increases, the exchange of air and metabolites and the supply of nutrients are restricted by simple diffusion, resulting in accumulation of ROS in microtissues, which may cause cell damage and death. The Example is intended to present the effect on ROS-mediated cell damage of constituent cells according to the size of the peripheral nerve-mimicking microtissue. PNSCs collected from the monolayer culture environment are suspended in a suspension culture medium at a density of 1.0E+06/mL. The suspension culture medium is prepared by adding 1% human serum albumin (HSA, Green Cross Corporation) in DMEM/F12 culture media. PNSCs seeded using ultra low attachment (ULA) culture vessels induce cell-to-cell bindings by preventing adhesion to the culture vessel. For PNSCs suspended in the suspension culture medium, 100, 200, 500, 750, 1000, 2500, and 5000 cells per well in a 96-well ULA culture vessel (SPL Life Sciences, Seoul, Korea) were seeded and then centrifuged at 500×g for 10 minutes to collect the cells in the center of the culture vessel, followed by culture for 24 hours. After 24 hours of suspension culture, a degree of ROS accumulation in microtissues is evaluated using a confocal scanning microscope using CM-H2DCFDA (Molecular Probe, Eugene, OR).

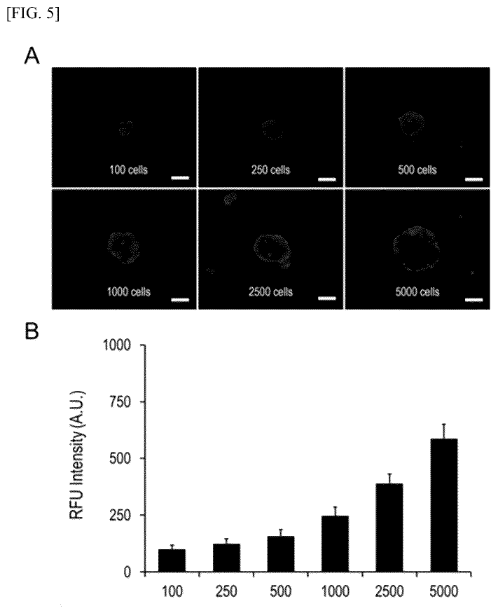

FIG. 5 shows results of radical oxygen species (ROS, free radicals, and active oxygen) accumulation according to a size of the microtissue. It may be observed that the fluorescence intensity, which indicates the degree of ROS accumulation, increases in proportion to the microtissue size. In particular, it may be found that ROS accumulation increases rapidly when the number of cells that make up the peripheral nerve-mimicking microtissue is more than 1000, and that stability of the microtissue may be secured by forming the microtissue less than 200 μm in diameter with the number of constituent cells less than 1000.

### Example 5

An increase in the size of microtissues may limit the supply of oxygen and nutrients by simple diffusion, resulting in the death of cells in the peripheral nerve-mimicking microtissues. The Example is intended to present an effect on cell death of constituent cells according to the size of the peripheral nerve-mimicking microtissue. PNSCs collected from the monolayer culture environment are suspended in a suspension culture medium at a density of 1.0E+06/mL. The suspension culture medium is prepared by adding 1% human serum albumin (HSA, Green Cross Corporation) in DMEM/F12 culture media. PNSCs seeded using ultra low attachment (ULA) culture vessels induce cell-to-cell bindings by preventing adhesion to the culture vessel. For PNSCs suspended in suspension culture medium, 100, 200, 500, 750, 1000, 2500, and 5000 cells are seeded in each well in a 96-well ULA culture vessel (SPL Life Sciences, Seoul, Korea), and then centrifuged at 500×g for 10 minutes to collect the cells in the center of the culture vessel, followed by culture for 24 hours. After 24 hours of suspension culture, the number of ethidium homodimer-1 (EthD-1, Molecular Probe) positive cells are calculated to assess cell death. Peripheral nerve-mimicking microtissues are imaged using a confocal scanning microscope to evaluate cell death using ImageJ.

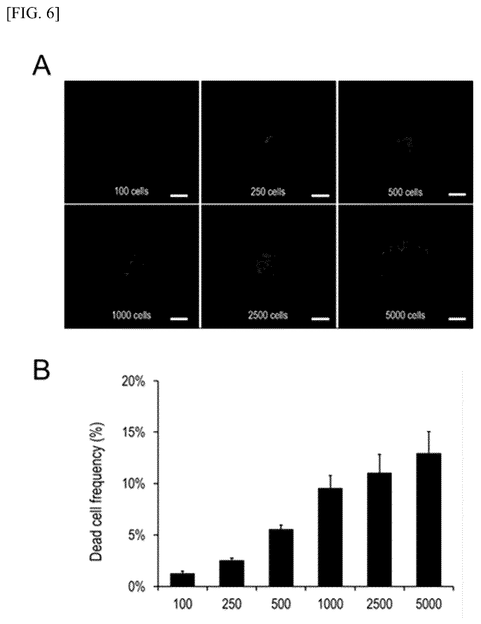

As presented in FIG. 6, it may be observed that cell death increases as the size of the peripheral nerve-mimicking microtissue increases. In a similar tendency to the ROS accumulation results, when the number of cells that make up the peripheral nerve-mimicking microtissue is more than 1000 while the size is more than 200 μm, cell death increases rapidly. Through the Example, it may be determined that the control of the number and size of constituent cells of microtissues is an important regulating factor in the manufacture of peripheral nerve-mimicking microtissues.

### Example 6

As a result of limitation in oxygen and nutrient exchange during the production process of peripheral nerve-mimicking microtissues, ROS accumulates in tissues, leading to cell damage and death. The Example is intended to present a cytoprotection method by dexamethasone (DEX) and N-acetylcysteine (NAC) to inhibit cell damage and death. PNSCs collected from the monolayer culture environment are suspended in a suspension culture medium at a density of 1.0E+06/mL. The suspension culture medium is prepared by adding 1% HSA in DMEM/F12 culture medium. 1.0×10⁵ cells per cm² area of a culture vessel is seeded in the ULA T75 flask (SPL Life Sciences) culture vessel. 1 mM NAC, 1 μM DEX, NAC and DEX were added simultaneously in 15 mL of suspension culture medium and cultured for 3 days. Cell death was assessed by LIVE/DEAD staining, and, for cell death, the survival rate was evaluated by calculating the total number of cells stained with DAPI and counting the number of cells that are positive for ethidium homodimer-1 (EthD-1). Moreover, the expression of p38 MAPK and cleaved caspase, which are factors that regulate cell death, and the degree of expression of p-Akt, an anti-cell death regulating factor, were evaluated through immunofluorescence staining.

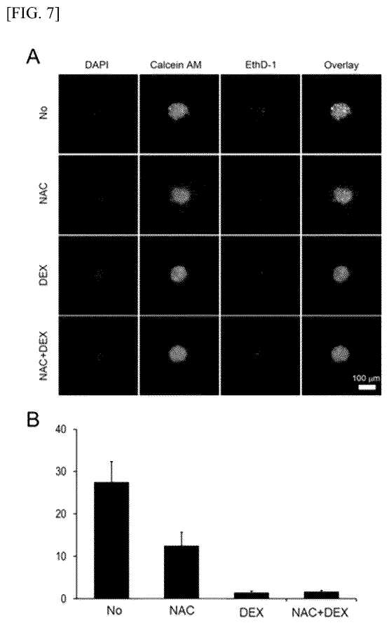

As presented in FIG. 7, the protective effect of NAC and DEX on constituent cells in the peripheral nerve-mimicking microtissue may be identified. It may be determined that the cytoprotective effect due to addition of DEX compared to NAC is great, and whether NAC is mixed did not significantly enhance a cytoprotective mechanism of DEX.

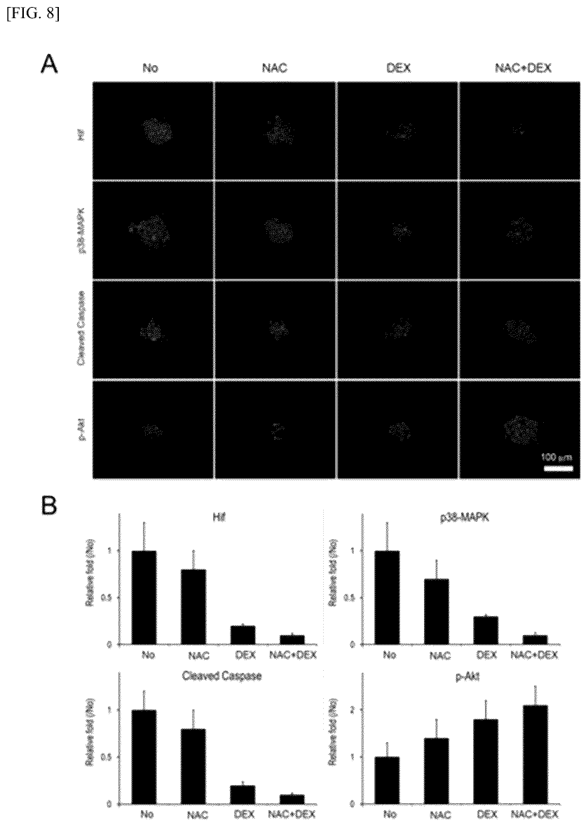

As presented in FIG. 8, NAC and DEX were able to significantly inhibit expression of Hif, p38 MAPK, and cleaved caspase, which are cell death proteins that cause cell damage and death. In particular, for DEX, when NAC was added to the suspension culture at the same time, in the comparison of expression of Hif, p38 MAPK, and cleaved caspase with single administration, high inhibitory ability may be observed. Moreover, expression of p-Akt, a cytoprotective protein, may be enhanced by co-administration of NAC and DEX. Through the Example, presented is a method to increase the cell survival rate by inhibiting cell death through the addition of low concentration DEX and NAC in the culture medium used in the suspension culture environment.

### Example 7

The Example presents a method of industrially mass-producing peripheral nerve-mimicking microtissues. PNSCs collected from the monolayer culture environment are suspended in a suspension culture medium at a density of 1.0E+06/mL. The suspension culture medium is prepared by adding 1% HSA, 1 μM DEX, and 1 mM NAC in DMEM/F12 culture media. Suspension culture is performed using ULA T75 flask (SPL Life Sciences) culture vessel. 1.0˜1.5×10⁵ cells per cm² area of the culture vessel are seeded in the culture vessel. 7.5˜11.1×10⁶ cells were seeded in ULA T75 culture vessel, and then 15 mL of suspension culture medium was added, followed by culture for 3 days.

For the prepared microtissue, paraffin tissue blocks were prepared in the conventional way, the 5 μm thick paraffin sections were thinned, and HE staining and immunofluorescence staining were performed to evaluate the structural characteristics. The accumulation of collagen type-IV and laminin, which are peripheral nerve-specific extracellular matrix in microtissues, was evaluated using immunofluorescence staining. Junctions of cell-to-cell and cell-to-extracellular matrix in microtissues were evaluated through expression of β-catenin and integrin-β1.

In order to evaluate the expression of mRNA related to the Wnt signaling pathway in microtissues, RNA was isolated from PNSC before suspension culture and peripheral nerve-mimicking microtissue after manufacturing, and cDNA was prepared using reverse transcription reaction. Gene expression was evaluated using a PCR microarray including a starter capable of amplifying Wnt signaling pathway regulating factors. Whether the Wnt signaling was activated was evaluated by expression of its target genes activated by Wnt receptors, ligands, and downstream Wnt pathway expression rates were expressed as the expression rate (fold) compared to PNSCs before microtissue formation.

As presented in FIG. 9, after 3 days of culture, it was possible to prepare 2,350±451 microtissues in a size of 86.5±27.3 μm in T75 flask. The microtissue has a structure in which PNSCs are bound, and it may be found through immunofluorescence staining that collagen type-VI and laminin which are peripheral nerve-specific ECMs accumulated in the microtissue, β-catenin and CD29 (integrin-β1), referring to cell-to-cell and cell-to-ECM bindings, were expressed uniformly in the microtissue, and the microtissue prepared through the culture process functions as a substrate to allow peripheral nerve ECMs to be produced and accumulated among intercellular spaces while binding with PNSCs, thereby securing structural stability by binding with cells. Moreover, it was presented that the cell-to-cell binding ability was strengthened by junction via β-catenin among dense cells, suggesting that it may be manufactured in a structure similar to tissues in vivo, rather than a simple cell aggregate.

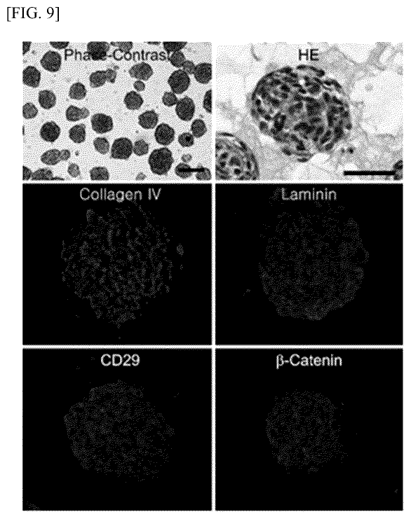

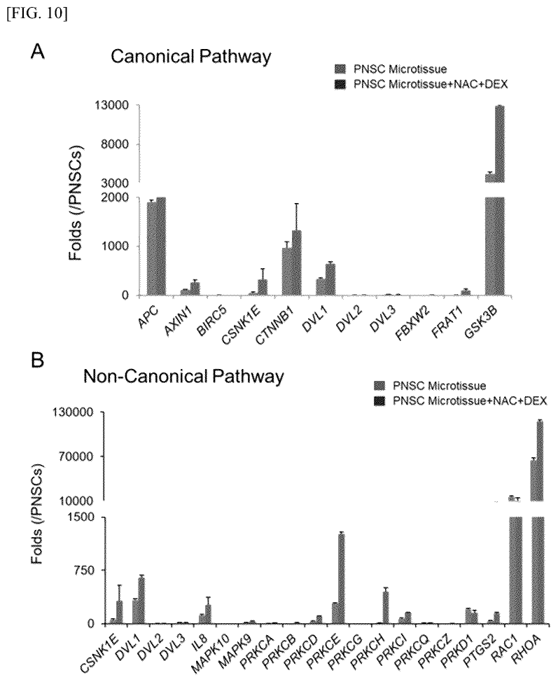

FIG. 10 presents activation of the Wnt/β-catenin signaling pathway through formation of peripheral nerve-mimicking microtissues. Of the Wnt pathways, both canonical and non-canonical signaling pathways would show how mRNA expression induces multifold to thousand-fold increases through microtissue formation. In particular, the addition of NAC and DEX in the manufacture of microtissues would show how to prevent cell death and induce an increase in mRNA expression. Through microtissue formation, it was possible to observe that the expression of APC, CTNB1, and GSK3B mRNA, which are pivotal regulatory genes in the canonical signaling pathway of PNSC, was increased by several to tens of times (Table 1 and Table 2). Table 1 shows the canonical Wnt pathway, and Table 2 shows the non-canonical Wnt pathway.

In tests that the Wnt signaling pathway was activated through microtissue formation and expression of receptors, ligands, and downstream target genes acting on Wnt signaling was investigated, gene expression increased several to tens of times compared to PNSCs prior to microtissue formation (Table 3, Table 4, and Table 5). Table 3 shows Wnt receptors, Table 4 shows Wnt ligands, and Table 5 shows Wnt targets. In terms of Wnt-downstream target genes, it may be found that expression of functional genes that regulate cell migration and differentiation significantly increases.

Through the Example, provided is a method capable of strengthening biological functions of PNSCs by controlling a PNSC density seeded in the industrialized mass production system, providing structural stability by protecting cells in microtissues through NAC and DEX addition, and at the same time activating Wnt signaling pathways through microtissue formation.

TABLE 1

| | PNSC | Spheroid | Fold(Spheroid/PNSC) |
|---|---|---|---|
| APC | 1900.3139 | 2333.7149 | 1.2 |
| AXIN1 | 112.0361 | 260.2215 | 2.3 |
| BIRC5 | 6.9589 | 1.1492 | 0.2 |
| CSNK1E | 52.931 | 321.2896 | 6.1 |
| CTNNB1 | 965.6604 | 1327.5161 | 1.4 |
| DVL1 | 330.5251 | 643.3648 | 1.9 |
| DVL2 | 2.6287 | 5.482 | 2.1 |
| DVL3 | 15.4632 | 12.8249 | 0.8 |
| FBXW2 | 0.0108 | 0.3777 | 35.0 |
| FRAT1 | 8.6761 | 101.1429 | 11.7 |
| GSK3B | 4243.0195 | 12878.1063 | 3.0 |

TABLE 2

| | PNSC | Spheroid | Fold(Spheroid/PNSC) |
|---|---|---|---|
| CSNK1E | 52.931 | 321.2896 | 6.1 |
| DVL1 | 330.5251 | 643.3648 | 1.9 |
| DVL2 | 2.6287 | 5.482 | 2.1 |
| DVL3 | 15.4632 | 12.8249 | 0.8 |
| IL8 | 120.9279 | 264.7322 | 2.2 |
| MAPK10 | 0.1484 | 0.6123 | 4.1 |
| MAPK9 | 12.8026 | 34.4333 | 2.7 |
| PRKCA | 5.3507 | 10.3244 | 1.9 |
| PRKCB | 0.2662 | 11.9818 | 45.0 |
| PRKCD | 33.4928 | 105.2814 | 3.1 |
| PRKCE | 285.5293 | 1258.1558 | 4.4 |
| PRKCG | 0.0089 | 0.3971 | 44.6 |
| PRKCH | 9.4718 | 447.7214 | 47.3 |
| PRKCI | 70.804 | 162.0045 | 2.3 |
| PRKCQ | 12.0648 | 11.9818 | 1.0 |
| PRKCZ | 0.1419 | 1.2774 | 9.0 |
| PRKD1 | 203.2655 | 153.1221 | 0.8 |
| PTGS2 | 42.1171 | 142.7001 | 3.4 |
| RAC1 | 15764.4184 | 7914.6079 | 0.5 |
| RHOA | 64903.4372 | 117195.4188 | 1.8 |

TABLE 3

| | PNS | Spheroid | Fold(Spheroid/PNSC) |
|---|---|---|---|
| FZD1 | 204.5757 | 3360.1308 | 16.4 |
| FZD10 | 1.8537 | 107.4156 | 57.9 |
| FZD2 | 5896.43 | 13595.2735 | 2.3 |
| FZD3 | 18.3182 | 202.3295 | 11.0 |
| FZD5 | 92.3045 | 676.7688 | 7.3 |
| FZD6 | 1209.123 | 135.63 | 0.1 |
| FZD7 | 9.9829 | 49.4148 | 4.9 |
| FZD8 | 585.1525 | 4581.9293 | 7.8 |
| FZD9 | 13.7685 | 193.1318 | 14.0 |
| LDLR | 3.5922 | 12.2162 | 3.4 |
| SFRP2 | 0.8996 | 64.5313 | 71.7 |
| SFRP4 | 2639.257 | 8483.251 | 3.2 |
| WISP1 | 168.1869 | 682.0261 | 4.1 |

TABLE 4

| | PNS | Spheroid | Fold(Spheroid/PNSC) |
|---|---|---|---|
| WNT1 | 0.6423 | 36.3692 | 56.6 |
| WNT10B | 0.178 | 2.7455 | 15.4 |
| WNT11 | 0.2695 | 4.5854 | 17.0 |
| WNT16 | 34.2178 | 176.5939 | 5.2 |
| WNT2 | 51.1929 | 151.1338 | 3.0 |
| WNT2B | 0.4351 | 33.7387 | 77.5 |
| WNT3 | 40.3338 | 137.5871 | 3.4 |
| WNT3A | 0.165 | 4.5435 | 27.5 |
| WNT4 | 0.307 | 2.3252 | 7.6 |
| WNT5A | 139.9511 | 122.5765 | 0.9 |
| WNT5B | 691.6817 | 701.3754 | 1.0 |
| WNT6 | 1.0135 | 47.6232 | 47.0 |
| WNT7A | 0.2662 | 11.9818 | 45.0 |
| WNT8A | 0.2415 | 9.2259 | 38.2 |
| WNT9A | 2.5123 | 7.8619 | 3.1 |

TABLE 5

| | PNS | Spheroid | Fold(Spheroid/PNSC) |
|---|---|---|---|
| BMP4 | 130.7912 | 119.2249 | 0.9 |
| CCND1 | 43.895 | 533.8579 | 12.2 |
| CCND2 | 0.416 | 3.132 | 7.5 |
| CCND3 | 46.8075 | 59.6201 | 1.3 |
| CD44 | 24231.49 | 73656.6354 | 3.0 |
| CDX1 | 0.875 | 78.9348 | 90.2 |
| CLDN1 | 5.3149 | 9.7041 | 1.8 |
| EDN1 | 511.3655 | 135.48 | 0.3 |
| EGFR | 1.8255 | 5.739 | 3.1 |
| FGF4 | 1.2354 | 58.9709 | 47.7 |
| FGF9 | 0.6377 | 39.8241 | 62.4 |
| FN1 | 5.4201 | 36.5258 | 6.7 |
| FOSL1 | 46.3549 | 65.9074 | 1.4 |
| HNF1A | 0.048 | 2.16 | 45.0 |
| ID2 | 2847.529 | 5549.1401 | 1.9 |
| JAG1 | 93.648 | 313.2219 | 3.3 |
| JUN | 1205.176 | 1179.7148 | 1.0 |
| MET | 2751.6 | 5615.5158 | 2.0 |
| MMP2 | 1084.906 | 4535.5575 | 4.2 |
| MMP9 | 0.2662 | 11.9818 | 45.0 |
| MYC | 1.5725 | 46.8211 | 29.8 |
| MYCN | 0.0063 | 0.283 | 44.9 |
| NANOG | 0.3851 | 18.9931 | 49.3 |
| NOS2 | 0.0152 | 0.6844 | 45.0 |
| PLAU | 352.8442 | 59.9311 | 0.2 |
| RUNX2 | 2970.474 | 10488.4195 | 3.5 |
| SOX2 | 1.3464 | 113.2633 | 84.1 |
| SOX9 | 95.4106 | 36.9588 | 0.4 |
| TT | 3.9043 | 27.8486 | 7.1 |
| VEGFA | 1.7192 | 151.0284 | 87.8 |

### Example 8

The Example presents structural characteristics and biological characteristics of peripheral nerve-mimicking microtissue mass-manufactured at the industrial level. PNSCs collected from the monolayer culture environment are suspended in a suspension culture medium at a density of 1.0E+06/mL. The suspension culture medium is prepared by adding 1% HSA, 1 μM DEX, and 1 mM NAC in DMEM/F12 culture media. Suspension culture is performed using ULA T75 flask (SPL Life Sciences) culture vessel. 1.5×10⁵ cells per cm² area of the culture vessel were seeded. 1.1×10⁷ cells were seeded in ULA T75 culture vessel, and then 15 mL of suspension culture medium was added, followed by culture for 3 days. Using the prepared microtissue, paraffin tissue blocks were prepared via a conventional method, then the 5 μm thick paraffin sections were thinned, and immunofluorescence staining was performed using neurons, neural crest cells, glial cells, Schwann cells, and myelin markers to evaluate the constituent cells.

In order to evaluate the expression of neurotrophic mRNA in microtissues, RNA was isolated from PNSC before suspension culture and peripheral nerve-mimicking microtissue after manufacturing, and cDNA was prepared using reverse transcriptase. Gene expression was evaluated using a PCR microarray consisting of a starter to amplify neurotrophic mRNA. Moreover, the mRNA expression of anti-inflammatory regulating factors and angiogenesis inducing factors of the microtissue was also evaluated through PCR microarray. Since the titers of neurotrophic protein secretion in microtissues mediate an important role in neuroregeneration, the content of BDNF, GDNF, IGF-1, IL-6, NGF, and NT-3 proteins, which are representative neurotrophic proteins in the culture medium, was analyzed through ELISA after the preparation of microtissue.

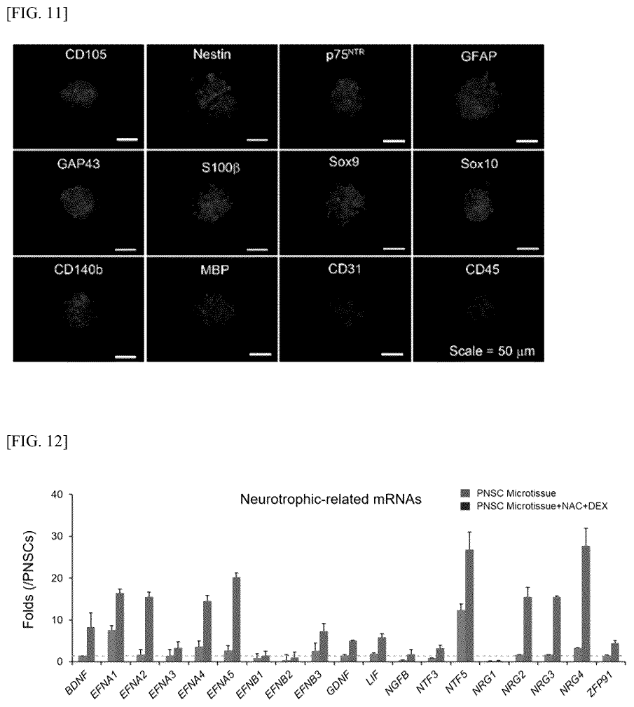

FIG. 11 shows characteristics of constituent cells in the peripheral nerve-mimicking microtissue manufactured by the mass production method. The constituent cells in the microtissue maintained the expression of CD105, nestin, and p75^{NTR}, which are neural crest-derived cell markers and had the characteristics similar to peripheral nerve-derived stem cells. However, after 3 days of culture, PNSCs in the microtissue showed expression of GFAP, GAP43, and S100B which were not expressed in the pre-culture state. This suggests that cells having differentiated into Schwann cells are mixed, and in particular, cells with Schwann progenitor cell characteristics in which Sox2 and Sox10 are co-expressed are composed. It was found that a composition includes myelinating Schwann cells that express MBP and interstitial stromal cells that express CD140b, which are composed of peripheral nerve-like cells that are regenerated after damage. Through the Example, provided is a method of manufacturing a microtissue similar to cells constituting peripheral nerves in the process of regeneration after damage, wherein a peripheral nerve-mimicking microtissue includes GFAP−/S100β−/Sox10+ undifferentiated neural crest cells, GFAP+/S100β+/myelin+ myelinated Schwann cells, GFAP+/GAP43+/myelin-repair Schwann cells, and GFAP−/CD140a+ interstitial stromal cells.

FIG. 12 shows characteristics of neurotrophic gene expression in peripheral nerve-mimicking microtissues manufactured via mass production. The expression of neurotrophic genes in mass-produced peripheral nerve-mimicking microtissues was evaluated via a PCR microarray method. Compared to expression of neurotrophic mRNA expressed in PNSCs in a stage before formation of microtissues, it is suggested that the expression of the corresponding gene is significantly increased after microtissue formation, and in particular, it is a method to induce a significant increase in gene expression through the addition of NAC and DEX during microtissue formation. The peripheral nerve-mimicking microtissue presents evidence for significant application to neuroregeneration via peripheral nerve-mimicking microtissue formation through a multi-to-tens increase in expression of neurotrophic family mRNAs including BDNF, NGF, neutrophin-3 and neurotrophin-4, ephrin family mRNAs, GDNF family mRNAs including GDNF and artemin, and CNTF family mRNA including IL-6, CNTF, and LIF that play a key role in neuroregeneration (Table 6).

As peripheral nerve-mimicking microtissues are formed, growth factors, immune response regulating factors, and angiogenesis inducing factor regulatory mRNA expression were tested. As shown in Table 7 to Table 9, it may be identified that microtissue formation results in remarkable increases in growth factors, immune response regulating factors, and angiogenesis inducing factor mRNAs compared to PNSCs before formation. In addition to neurotrophic factors, the expression of EGF, FGF, and IGF-1 was significantly increased (Table 7), the expression of IL10 mRNA, a key cytokine capable of regulating excessive inflammatory responses, was increased by more than 50 times (Table 8), the angiogenesis inducing factors ANGPT, EPNA, EPO, PDGF, and VEGF mRNAs were significantly increased, and the biological functions of PNSCs may be enhanced and activated through microtissue formation (Table 9).

TABLE 6

| | Fold(Microtissue/PNSC) |
|---|---|
| BDNF | 8.329545 |
| EFNA1 | 16.45205 |
| EFNA2 | 15.57143 |
| EFNA3 | 3.375 |
| EFNA4 | 14.52727 |
| EFNA5 | 20.2125 |
| EFNB1 | 1.557175 |
| EFNB2 | 1.066362 |
| EFNB3 | 7.292308 |
| CTNF | 21.34286 |
| GDNF | 5.100746 |
| LIF | 5.871126 |
| NGFB | 1.803059 |
| NTF3 | 3.224868 |
| NTF5 | 26.88 |
| NRG1 | 0.247363 |
| NRG2 | 15.57143 |
| NRG3 | 15.57143 |
| NRG4 | 27.74699 |
| ZFP91 | 4.514059 |

TABLE 7

| | Fold(Microtissue/PNSC) |
|---|---|
| EGF | 5.7782 |
| FGF1 | 2.7127 |
| FGF2 | 5.2133 |
| FGF4 | 14.9951 |
| FGF5 | 1.5709 |
| FGF6 | 15.3838 |
| FGF7 | 9.1643 |
| FGF8 | 10.9971 |
| FGF9 | 10.6728 |
| FGF10 | 15.3838 |
| FGF11 | 13.6358 |
| FGF12 | 25.8563 |
| FGF13 | 14.3585 |
| FGF14 | 15.3838 |
| FGF16 | 56.8739 |
| FGF18 | 23.3294 |
| FGF19 | 79.1608 |
| FGF20 | 64.587 |
| FGF23 | 47.6554 |
| IGF1 | 34.2081 |
| GAS6 | 8.707 |

TABLE 8

| | Fold(Microtissue/PNSC) |
|---|---|
| CLC | 86.1526 |
| CTF1 | 9.338 |
| CSF1 | 0.697 |
| CSF2 | 5.3725 |
| CSF3 | 18.4823 |
| GHI | 43.778 |
| GH2 | 11.9183 |
| FLT3LG | 4.5242 |
| IDO1 | 45.2907 |
| IL2 | 13.5486 |
| IL3 | 3.8065 |
| IL5 | 15.3838 |
| IL7 | 6.0954 |
| IL9 | 20.6523 |
| IL10 | 58.9269 |
| IL11 | 3.8823 |
| IL12A | 2.6021 |
| IL12B | 51.4272 |
| IL15 | 2.2373 |
| IL20 | 5.4343 |
| IL21 | 12.7715 |
| IL22 | 9.6094 |
| IL23A | 15.8498 |
| IL24 | 8.208 |
| IL26 | 35.0665 |
| IL28A | 10.5773 |
| IL29 | 30.2858 |
| IFNA1 | 49.6315 |
| IFNB1 | 66.5053 |
| IFNW1 | 19.4241 |
| IFNK | 33.2062 |
| IFNE1 | 44.3668 |
| IFNG | 94.7818 |
| KITLG | 4.7685 |
| LEP | 47.4641 |
| PRL | 177.0349 |
| TGFB | 32.6947 |
| TPO | 15.3838 |
| TSLP | 60.2545 |

TABLE 9

| | Fold(Microtissue/PNSC) |
|---|---|
| ANGPT1 | 15.57143 |
| ANGPT2 | 2.190476 |
| ANGPT4 | 3.507289 |
| EFNA1 | 16.45205 |
| EFNA2 | 15.57143 |
| EFNA3 | 3.375 |
| EFNA4 | 14.52727 |
| EFNA5 | 20.2125 |
| EFNB1 | |
| EFNB2 | |
| EFNB3 | 7.292308 |
| EPO | 9.95302 |
| PDGFC | 2.372724 |
| PDGFD | 9.25 |
| VEGFA | 3.169355 |
| VEFGB | 5.845967 |
| VEGFC | 3.172725 |

### Example 9

The Example evaluated cell damage by ROS in mass-manufactured peripheral nerve-mimicking microtissues. Microtissues and PNSCs composed of the same number of cells (1.0E+07 cells) were subjected to a reaction with 100 nM sodium arsenite for 1 hour to induce ROS cell damage. After inducing sodium arsenite-mediated cell damage, PNSCs and microtissues were treated with RIPA buffer to obtain cell lysates. Through PAGE, cell lysates were electrophoresed and then transferred to the PVDF membrane, and the expression of the cell death regulating proteins p-c-Jun, p-p38MAPK, p-MAPKAPK-2, p-JNK, and cleaved caspase 3 was evaluated. In order to analyze the semi-quantitative expression rate, the density of the band was measured through an image analyzer (Image J) to compare the expression rate. Moreover, the survival rate of PNSCs and microtissues after sodium arsenite treatment was analyzed by evaluating LIVE/DEAD staining and the expression rate of annexin V.

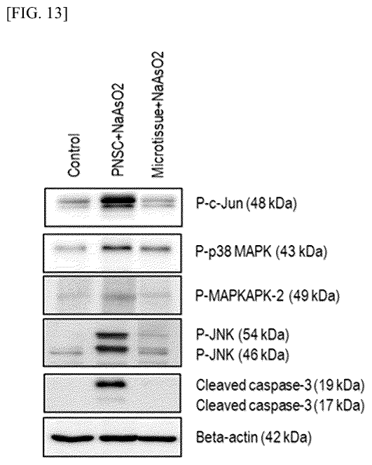

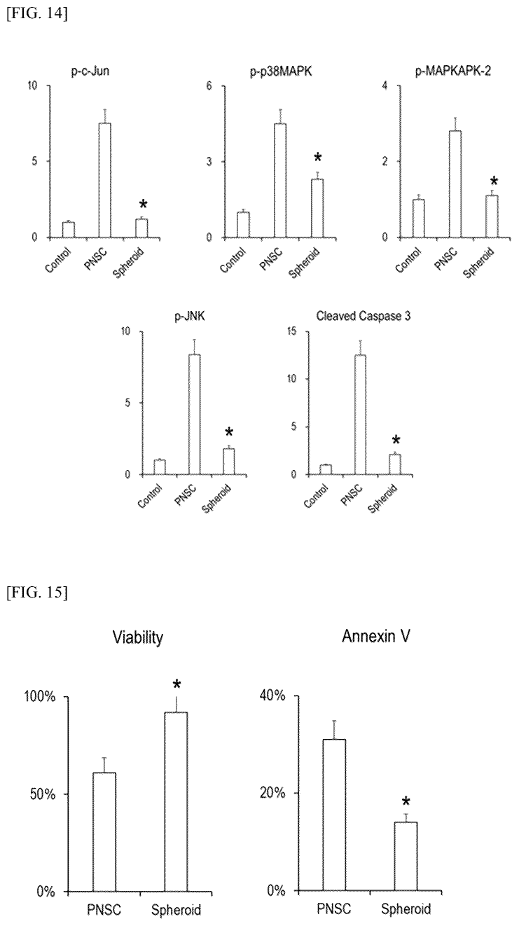

As presented in FIG. 13, it was found that sodium arsenite induced cell death of PNSCs and microtissues. Treatment with sodium arsenite showed a significant increase in the expression of p-c-Jun, p-p38MAPK, p-MAPKAPK-2, p-JNK, and cleaved caspase 3, which mediate cell death. On the other hand, in microtissues, it was found that the expression of p-c-Jun, p-p38MAPK, p-MAPKAPK-2, p-JNK, and cleaved caspase 3, which were significantly lower than PNSCs, was significantly reduced. In the results of comparing the expression rate of cell death regulating factors by densitometer (FIG. 14), the expression of cell death factors was significantly lower than PNSCs compared to microtissues (p<0.01). After inducing cell death through sodium arsenite treatment, the cell survival rate was quantitatively evaluated by LIVE/DEAD staining and annexin V expression, and it was found that significantly high cell survival rate and low annexin V expression were observed in microtissues compared to PNSCs with a tendency similar to the expression of cell death regulating factors (FIG. 15, p<0.01).

The above results provide a method of enhancing resistance to ROS compared to PNSCs through microtissue formation.

### Example 10

The Example evaluated the secretion levels of neurotrophic factors from mass-manufactured peripheral nerve-mimicking microtissues. Stem cell therapeutics are known to have an indirect mechanism that is expected to be effective by substances secreted by stem cells that are administered. The regeneration of damaged nerve tissue requires mobilization and growth of neural stem cells and the axon regrowth, as well as the efficacy of suppressing the excessive inflammatory response induced after damage, and a mechanism capable of promoting regeneration through vascular regeneration in the damaged tissue is needed.

The neurotrophic proteins were evaluated by measuring neurotrophic proteins in conditioned media collected during manufacturing of PNSCs and microtissues culture. The content of BDNF, GDNF, IGF-1, IL-6, NGF, and NT3, which are representative neurotrophic proteins in the conditioned medium, was measured via ELISA. Bone marrow-derived mesenchymal stem cells (BMSCs) were used as a control group to comparatively evaluate the ability to secrete neurotrophic proteins. The neurotrophic efficacy of substances secreted from PNSCs and microtissues was evaluated on a cell-based basis, and SH-SY5Y, a neural crest-derived neural stem cell line, was used.

The ability to induce neural stem cell growth was analyzed through dsDNA content analysis after the addition of conditioned media, and the dsDNA content of the increased cells was comparatively evaluated in percentage based on the dsDNA content before culture. The neuroregenerative efficacy was comparatively evaluated by measuring the length of neurite outgrowth of SH-SY5Y by the addition of conditioned media using an image analysis.

The anti-inflammatory efficacy of PNSCs and microtissues were evaluated based on RAW264.7 cells. RAW264.7 cells were sensitized with 100 ug of LPS, and after 6 hours, the content of TNF-α and IL-1B secreted by RAW264.7 in the culture medium was analyzed by ELISA method. By adding conditioned media obtained from PNSCs or microtissues during LPS sensitization, the anti-inflammatory efficacy was evaluated with a degree of inhibiting the secretion level of inflammatory cytokines in RAW264.7 cells by the conditioned media.

The ability to induce angiogenesis of PNSCs and microtissues was evaluated based on HUVEC cells. The ability to induce angiogenesis was comparatively evaluated by assessing the ability to induce HUVEC cell growth and inhibit sodium arsenite-mediated cell death by adding conditioned media obtained from PNSCs or microtissues.

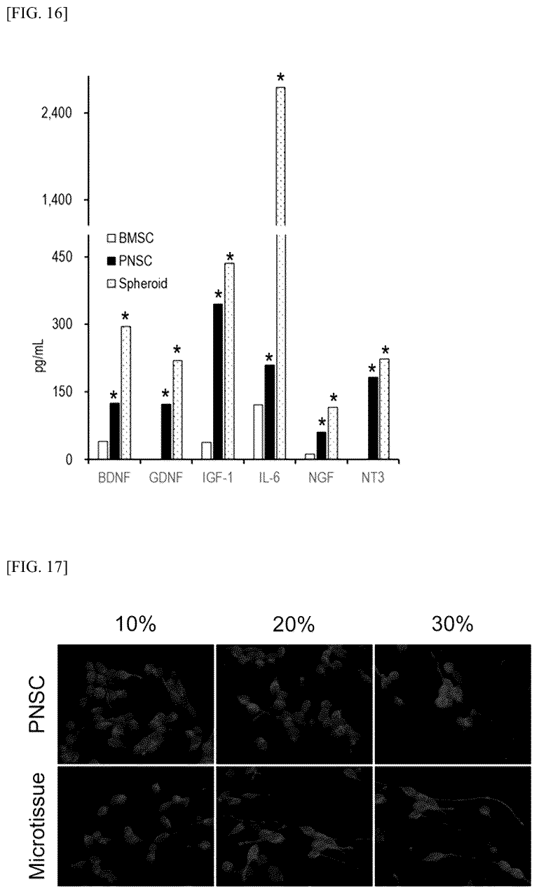

It was found that neurotrophic protein secretion ability of PNSCs was enhanced through microtissue formation with a tendency similar to neurotrophic mRNA expression (FIG. 16). Compared to bone marrow-derived mesenchymal stem cells (BMSCs), PNSCs had significantly higher secretion ability for neurotrophic proteins BDNF, GDNF, IGF-1, IL-6, NGF, and NT-3 (p<0.01). Moreover, it was found that the neurotrophic protein secretion ability of PNSCs may be remarkably enhanced through microtissue formation. Secretion of all neurotrophic proteins tested was significantly higher in the microtissues compared to PNSCs (P, 0.01).

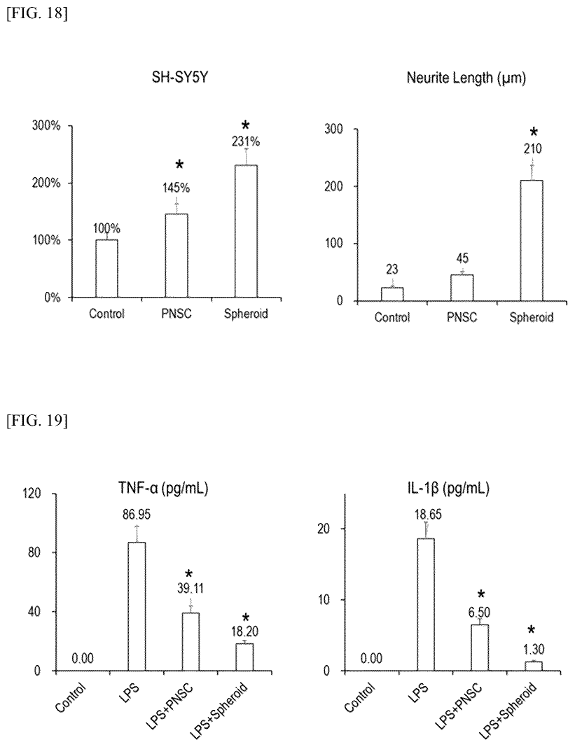

Neurotrophic activity, anti-inflammatory, and angiogenesis inducing titers were comparatively evaluated by cell-based analysis using conditioned media obtained from the microtissues and PNSCs. As presented in FIG. 17, the neurite outgrowth of neural stem cell increased when conditional media obtained from PNSCs or microtissues were added, and neurite length showed dependence on the concentration of the added conditional medium. Quantitative analysis has shown that the addition of PNSC and microtissue conditioned media significantly increases the neurite length of neural stem cells (FIG. 18, p<0.01). Moreover, the ability to induce the neurite outgrowth of neural stem cells through the addition of conditioned media was evaluated. It was possible to induce cell growth of neural stem cells in both PNSCs and microtissue conditioned medium (FIG. 18). In particular, it could be found that the conditioned media obtained from the microtissues showed significant neural stem cell growth and axon growth compared to the PNSCs conditioned medium (p<0.01).

In FIG. 19, it was possible to identify the anti-inflammatory efficacy of PNSCs and microtissues. TNF-α and IL-1B secretion was significantly increased in RAW264.7 cells sensitized with LPS, and it was possible to find that conditioned media obtained from PNSCs and microtissues showed anti-inflammatory efficacy of suppressing inflammatory cytokine secretion derived from sensitized cells. In particular, compared to PNSCs, the conditioned medium derived from microtissues showed significantly high ability of suppressing secretion of inflammatory cytokine (p<0.01), thereby deriving a result that high anti-inflammatory efficacy may be secured through microtissue formation.

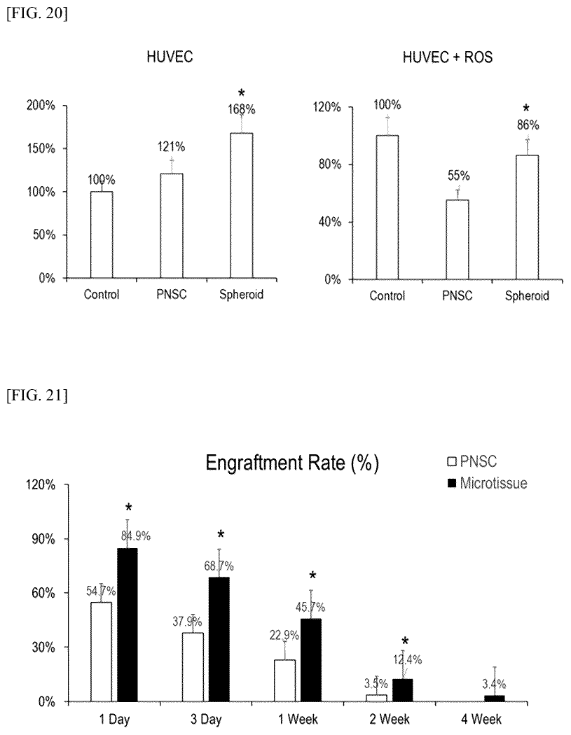

The ability to induce angiogenesis in PNSCs and microtissues was evaluated through HUVEC cell-based analysis, the significant growth of vascular endothelial cells may be induced through addition of conditioned media obtained from PNSCs and microtissues, and the ability to induce angiogenesis was identified by finding the significant protective ability of ROS-mediated HUVEC cells (FIG. 20). In particular, the addition of microtissue-derived conditioned media compared to PNSCs showed significant cell growth (p<0.01) of HUVEC, a vascular endothelial cell compared to PNSC conditioned media as well as a significant cytoprotective effect against ROS-mediated cell damage (p<0.01).

As a result above, the neurotrophic, anti-inflammatory, and angiogenesis inducing efficacy of PNSCs may be identified, and the neurotrophic, anti-inflammatory, and neovascularization inducing titers acting on angiogenesis of PNSCs were enhanced by formation of microtissue from PNSCs.

### Example 11

The Example evaluated the survival rate and efficacy after in vivo transplantation of mass manufactured peripheral nerve-mimicking microtissues. The spinal cord in the 7th and 8th thoracic vertebrae of the nude mouse was compressed to induce damage, and 3 days after the damage, 1.0E+05 PNSCs or microtissues composed of the same cells were injected into the spinal cord. Residual rate in the spinal cord after administration of PNSCs or microtissues was evaluated by qPCR targeting human specific Alu genes. The content of human-specific neurotrophic proteins BDNF, GDNF, IGF-1, NGF, and NT-3 in the spinal cord was analyzed via ELISA through administration of PNSCs and microtissues. The neuroregenerative efficacy of administered PNSCs or microtissues was evaluated by analyzing myelin regeneration and axon growth in the center of the affected area via morphometry.

As presented in FIG. 21, PNSCs and microtissues administered into injured the spinal cord tended to decrease proportionally over time after administration. Compared to PNSCs, when the microtissues were administered, the cell residual rate over all periods tested was significantly high (p<0.01). In the case of PNSCs, only 5% of cells remained after 2 weeks of administration, with no cells remained 4 weeks after administration. On the other hand, when the microtissues were administered, it was investigated that 12.4% of microtissues remained 2 weeks after administration, and 3.4% remained even 4 weeks after administration. The above results experimentally showed that the resistance to ROS was significantly higher in microtissues than PNSCs and that resistance to cell damage was also operational in vivo and showed a high survival rate.

A correlation between the number of residual cells and the titers of the cell is widely known. It is expected that indirect efficacy mediated by effective factors secreted by cells is predominant in relation to neuroregenerative efficacy of PNSCs or microtissues, rather than direct mechanism. The results of evaluating the content of neurotrophic factors in the spinal cord a week after administration of PNSCs or microtissues are presented in FIG. 22. Neurotrophic proteins analyzed in the spinal cord administered with microtissues were found to be significantly high with a tendency similar to the residual rate (p<0.01). Compared to PNSCs, when administered with microtissues, BDNF, GDNF, IGF-1, IL-6, NGF, and NT-3 were all found to be in high content, and there was evidence for the neuroregenerative efficacy by these factors.

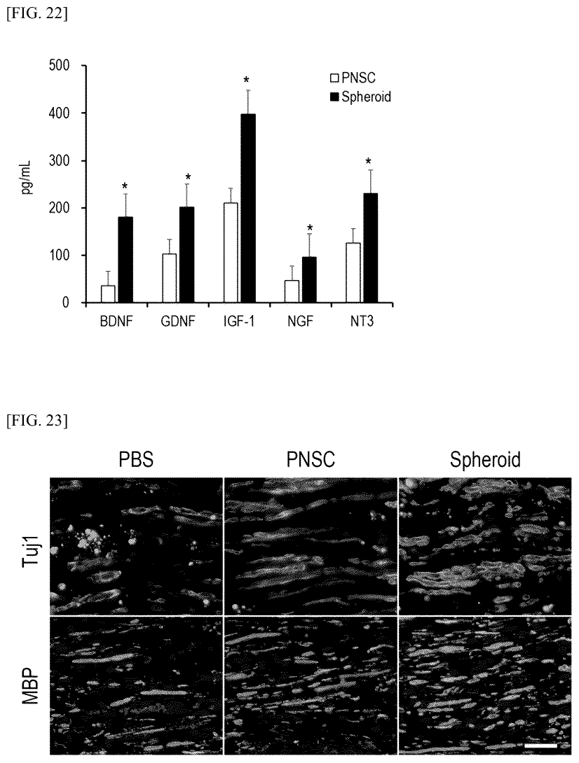

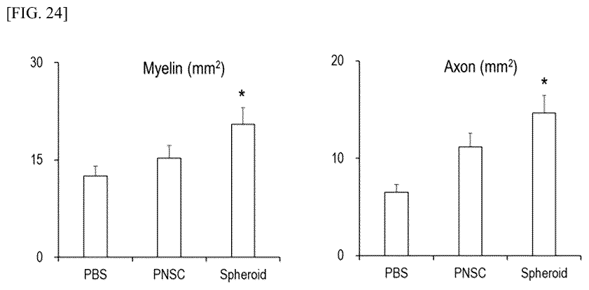

In the neuroregeneration after spinal cord damage, it is expected that the neural network is reformed through myelin regeneration and axon regrowth in the epicenter of the injured area, thereby restoring the motor and sensory functions of the spinal cord. Through the Example, it was possible to identify myelination and axon regrowth compared to animals that were not administered through PNSCs or microtissue administration, indicating that neuroregeneration would be enhanced through the administered cells (FIG. 23). In particular, compared to PNSCs, when the microtissue was administered, myelination and axon regrowth were significantly high (FIG. 24, p<0.01), showing high neuroregenerative efficacy of the microtissues.

The above results suggest that it is a method in which structural stability and functional titers may be enhanced through microtissues to improve residual rate and titer after transplantation in vivo, thereby increasing neuroregenerative efficacy.

1. A method of manufacturing a peripheral nerve-mimicking microtissue, the method comprising:
1) monolayer-culturing peripheral nerve-derived stem cells (PNSCs); and
2) collecting the monolayer-cultured PNSCs to be subjected to suspension culture in a culture medium comprising human serum albumin (HSA), dexamethasone (DEX), and N-acetylcysteine (NAC).

2. The method of claim 1, wherein, in the suspension culturing, 0.25 to 2.5×10⁵ PNSCs per cm² area of a culture vessel are seeded.

3. The method of claim 1, wherein the culture medium comprises 0.01 to 1% HSA, 0.1 to 5 μM DEX, and 0.1 to 10 mM NAC.

4. The method of claim 1, wherein the suspension culture induces cell-to-cell bindings of the PNSCs.

5. The method of claim 1, wherein the peripheral nerve-mimicking microtissue is a spheroid cell structure with 100 to 500 PNSCs bound together, and has a diameter of 100±20 μm.

6. A peripheral nerve-mimicking microtissue which is a spheroid cell structure with 100 to 500 PNSCs bound together that are subjected to suspension culture in a culture medium comprising HSA, DEX, and NAC, has a diameter of 100±20 μm, and is formed via cell-to-cell bindings of PNSCs and PNSC-to-extracellular matrix (ECM) bindings.

7. The peripheral nerve-mimicking microtissue of claim 6, wherein the peripheral nerve-mimicking microtissue has an extracellular matrix in which collagen type-VI and laminin produced and secreted from PNSCs are accumulated in intercellular spaces and cells are bound to extracellular matrix by CD29 and cells are bound to cells by β-catenin.

8. The peripheral nerve-mimicking microtissue of claim 6, wherein the peripheral nerve-mimicking microtissue comprises a peripheral nerve-derived adult stem cell, a Schwann progenitor cell, a repair Schwann cell, a myelinating Schwann cell, and a mesenchymal interstitial stromal cell.

9. The peripheral nerve-mimicking microtissue of claim 8, wherein the peripheral nerve-mimicking microtissue comprises a GFAP−/S100β−/Sox10+ undifferentiated neural crest cell, a GFAP+/S100β+/myelin+ myelinated Schwann cell, a GFAP+/GAP43+/myelin-repair Schwann cell, and a GFAP−/CD140a+ interstitial stromal cell.

10. The peripheral nerve-mimicking microtissue of claim 6, wherein the peripheral nerve-mimicking microtissue has a Wnt/β-catenin signaling pathway activated.

11. The peripheral nerve-mimicking microtissue of claim 6, wherein the peripheral nerve-mimicking microtissue has increased expression of any one or more neurotrophic factors selected from the group consisting of BDNF, EFNA1, EFNA2, EFNA3, EFNA4, EFNA5, EFNB1, EFNB2, EFNB3, CTNF, GDNF, LIF, NGFB, NTF3, NTF5, NRG1, NRG2, NRG3, NRG4, and ZFP91; any one or more growth factors selected from the group consisting of EGF, FGF1, FGF2, FGF4, FGF5, FGF6, FGF7, FGF8, FGF9, FGF10, FGF11, FGF12, FGF13, FGF14, FGF16, FGF18, FGF19, FGF20, FGF23, IGF1, and GAS6; any one or more immune response regulating factors selected from the group consisting of CLC, CTF1, CSF1, CSF2, CSF3, GH1, GH2, FLT3LG, IDO1, IL2, IL3, IL5, IL7, IL9, IL10, IL11, IL12A, IL12B, IL15, IL20, IL21, IL22, IL23A, IL24, IL26, IL28A, IL29, IFNA1, IFNB1, IFNW1, IFNK, IFNE1, IFNG, KITLG, LEP, PRL, TGFB, TPO, and TSLP; or any one or more angiogenesis inducing factors selected from the group consisting of ANGPT1, ANGPT2, ANGPT4, EFNA1, EFNA2, EFNA3, EFNA4, EFNA5, EFNB3, EPO, PDGFC, PDGFD, VEGFA, VEGFB, and VEGFC.

12. A method of treating nerve damage diseases, comprising:
administering a pharmaceutical composition comprising the peripheral nerve-mimicking microtissue of claim 6 as an active ingredient to a subject.

13. The method of claim 12, wherein the pharmaceutical composition promotes regeneration of nerve tissues.

14. (canceled)
